(19) United States
(12) Patent Application Publication
(10) Pub. No.: US 2013/0110106 A1
(43) Pub. Date: May 2, 2013

(54) EXPANDABLE STRUCTURE FOR OFF-WALL ABLATION ELECTRODE

(71) Applicant: Boston Scientific Scimed, Inc., Maple Grove, MN (US)

(72) Inventor: LEONARD B. RICHARDSON, Minneapolis, MN (US)

(73) Assignee: BOSTON SCIENTIFIC SCIMED, INC., MAPLE GROVE, MN (US)

(21) Appl. No.: 13/661,840

(22) Filed: Oct. 26, 2012

Related U.S. Application Data
(60) Provisional application No. 61/552,864, filed on Oct. 28, 2011.

(51) Int. Cl. A61B 18/14

(52) U.S. Cl. 606/41

(57) ABSTRACT

Catheter systems and methods for making and using catheters systems are disclosed. An example catheter system may include a catheter having a proximal end, and a distal end, an electrode for tissue modulation disposed on a distal portion of the catheter. An expandable basket structure may be placed over a distal section of the catheter. The expandable basket structure may have a basket having a fixed end and a movable end. The movable end may be slidably coupled to the catheter. An actuating structure may be connected to the movable end for moving the expandable basket between a collapsed state and an expanded state.

### CROSS-REFERENCE TO RELATED APPLICATIONS

This application claims priority under 35 U.S.C. §119 to U.S. Provisional Application Ser. No. 61/552,864, filed Oct. 28, 2011, the entirety of which is incorporated herein by reference.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices such as catheter systems. One example embodiment pertains to a catheter system for insertion in a patient's vasculature. The system includes a catheter and an electrode for tissue modulation or ablation disposed on a distal portion of the catheter. An expandable basket structure may be placed over a distal section of the catheter, the expandable basket structure having a basket having a fixed end and a movable end, wherein the movable end is slidably coupled to the catheter and an actuating structure connect to the movable end for moving the expandable basket between a collapsed state and an expanded state.

The basket may be proximal or distal the actuating structure. The actuating structure may be a spring. The spring may be biased to a shortened configuration or may be biased to an elongated configuration. The system may include a pull wire attached to and extending proximally from the movable end of the basket. The pull wire may be held in tension to hold the actuating structure in a shortened configuration until released to expand the basket or may apply tension once the distal end of the system is in place to deploy the basket. Other embodiments may include a sheath extending over the basket to retain the basket in the collapsed state. The sheath may be moved proximally relative to the catheter to release the basket to allow the actuating structure to expand the basket. The basket may expand symmetrically to help position the electrode within the center of a blood vessel or may expand asymmetrically to position the electrode closer to one side of a blood vessel. The basket may be disposed around the electrode. The electrode may be cylindrical and surround a portion of the catheter. The basket may comprise a non-metallic material such as a polymer, a ceramic, a glass or a composite such as a carbon fiber composite.

Another example embodiment pertains to a catheter system for insertion in a patient's vasculature. The system includes a catheter and an ablation electrode disposed at the distal end of the catheter. An expandable basket structure may be placed over a distal section of the catheter or over the electrode. The expandable basket structure may have a fixed end affixed to the catheter and a movable end attached to a first end of an actuation element. The actuation element has a second end affixed to the catheter and is configured to move the expandable basket between a collapsed state and an expanded state. The actuation element may comprise a mechanical structure such as a spring, a spring coil, a pneumatic structure, a closed cell foam, or an electrical or magnetic element configured to transform between a state of mechanical displacement and a state of energy storage.

Another example embodiment pertains to a method for ablating a nerve, the method comprising the steps of advancing an ablative catheter intravascularly to a location proximal of a nerve, the ablative catheter including a catheter shaft, an electrode and an expandable non-metallic basket extending over a section of the catheter; deploying the expandable basket structure in an expanded position in a vessel lumen such that portions of the basket structure contact the walls of the vessel lumen, and the bare electrode maintains a distance from the walls of the vessel lumen; and activating the bare electrode to ablate at least a portion of the nerve. The catheter may be any of the embodiments described herein. The ablating method may be radio frequency ablation, ultrasonic ablation or another suitable method. The vessel may be a renal artery. The expandable basket may be deployed by releasing a pull wire held in tension or by moving a sheath proximally from over the basket.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The accompanying drawings, which are incorporated in and constitute a part of this specification, illustrate embodiments of this disclosure and together with the description, serve to explain the principles of this disclosure.

FIGS. 1A and 1B illustrate a side view of an example ablation catheter.

FIGS. 2A and 2B illustrate a side view of another example ablation catheter.

FIGS. 3A and 3B illustrate a side view of another example ablation catheter.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

While the devices and methods described herein are discussed relative to renal nerve modulation, it is contemplated that the devices and methods may be used in other applications where nerve modulation and/or ablation are desired. In some instances, it may be desirable to ablate perivascular renal nerves with deep target tissue heating. As energy passes from an electrode to the desired treatment region, however, the energy may heat the fluid (e.g. blood) and tissue as it passes. As more energy is used, higher temperatures in the desired treatment region may be achieved thus resulting in a deeper lesion. However, this may result in some negative side effects, such as, but not limited to thermal injury to the vessel wall, blood damage, clotting and/or protein fouling of the electrode. Positioning the electrode away from the vessel wall may provide some degree of passive cooling by allowing blood to flow past both the electrode and the vessel wall.

Some approaches have used a self-expanding insulated metal basket to elastically self-deploy. However, it may be desirable to use lower-profile or non-metallic cage structures, but they may not have sufficient elastic strength to reliably self-deploy. The present approach utilizes a separate elastic structure to deploy the basket, so the basket itself does not need to have high elastic strength.

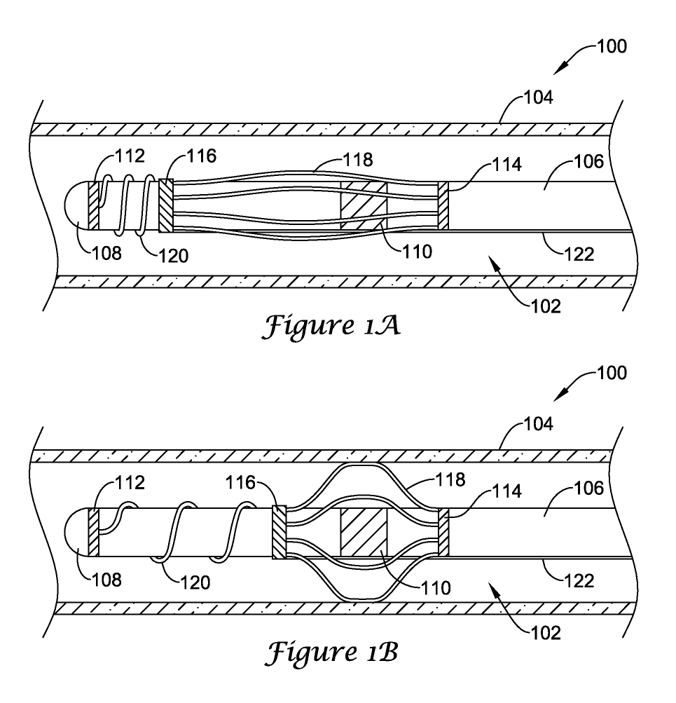

FIGS. 1A and 1B shown an illustrative embodiment of a renal nerve modulation system 100 disposed within a body lumen 102 having a vessel wall 104. The system 100 includes an elongate catheter shaft 106 having a distal end region. The elongate shaft 106 extends from a distal end 108 to a proximal end (not shown) configured to remain outside of a patient's body.

The shaft 106 further includes one or more electrodes 110. The electrode 110 may include an RF electrode. Accordingly, the electrode 110 may be configured to operate at a frequency of approximately 460 kHz. But it is contemplated that any suitable frequency in the RF range may be used, for example, from 100-500 kHz. However, it is contemplated that in other embodiments having different types of energy delivering structures different types of energy outside the RF spectrum may also be used, for example, but not limited to ultrasound, microwave, and laser.

While only a single electrode 110 is shown, embodiments are contemplated that include more than one (e.g., two, three, four, five, six, seven, eight, or more) electrodes 110. The electrodes 110 may be disposed at a suitable location along the catheter shaft 106.

To reduce the likelihood of close proximity of the electrode 110 to the vascular walls 104, the electrode 110 is spaced from the walls by means of an expansion system.

The system may include a fixed first attachment point 112 and a fixed second attachment point 114. In at least some embodiments, the first attachment point 112 may be positioned towards the distal end 108 of the shaft 106. Further, a movable collar 116 is disposed between the first and the second fixed attachment points 112/114. In the embodiment shown in the FIGS. 1A and 1B, an expandable basket 118 is defined or otherwise disposed between the fixed attachment point 114 and the collar 116. The expandable basket 118 may include wires or struts extending between the fixed attachment point 114 and the collar 116. The basket 118 illustrated in this embodiment includes straight wires but in other embodiments, basket 118 may comprise helical wires, a woven structure or other suitable expansion configuration. The basket 118 may expand radially when the collar 116 is moved towards the fixed attachment point 114 and may collapse when the collar 116 is moved away from the fixed attachment point 114. The basket 118 is thus movable between an expanded state and a collapsed state. The illustrated basket 118 expands symmetrically placing the electrode 110 at the center of the vasculature. In other embodiments (not shown), the basket expands asymmetrically to position the electrode closer to one side of a vascular wall 104.

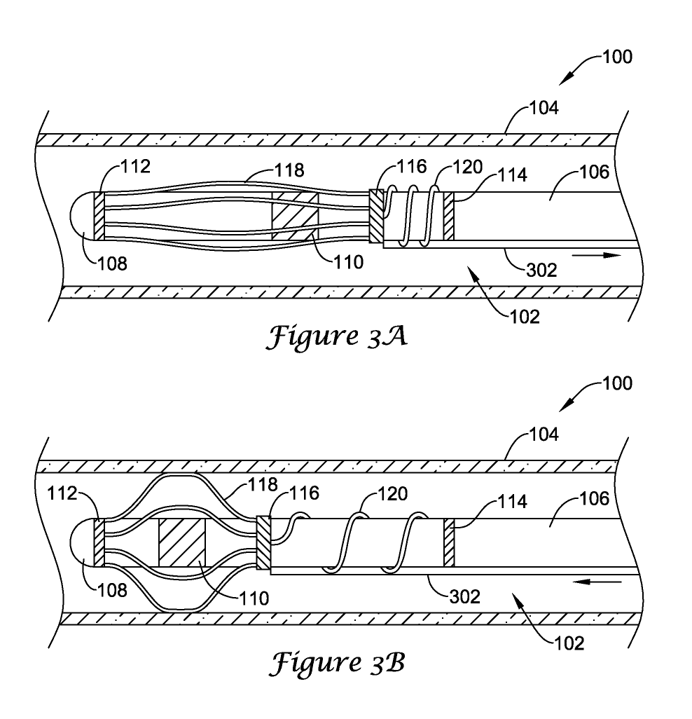

In the embodiment shown in FIGS. 1A and 1B, the expandable basket 118 is proximal an elastic element 120, which is attached to the fixed attachment point 112 and the collar 116. In FIGS. 1A and 1B the connection point between the elastic element 120 and the collar 116 is not explicitly seen but can be understood to be present on the opposite side of the catheter shaft 106. In other embodiments, the expandable basket 118 may be distal the elastic element 120 (e.g., as in FIGS. 3A and 3B).

In at least some embodiments, the elastic element 120 may take the form of a spring. In other embodiments (not shown), other suitable mechanisms may be used as the elastic element. For example, the elastic element 120 may be a rubber, a resilient polymer, a pneumatic element, a closed-cell foam, an element that converts stored electrical or magnetic energy into mechanical displacement or other suitable element. In the embodiment shown in FIGS. 1A and 1B, the elastic element 120 is attached at one end to the first fixed attachment point 112 and at another end to the collar 116, so that the expandable basket 118 is disposed between the collar 116 and second attachment point 114. In alternate embodiments (not shown), the elastic element 120 can be attached at one end to the second attachment point 114, and attached at another end to the movable attachment point 116, so that the expandable basket 118 is disposed between the movable attachment point 116 and first attachment point 112. The system 100 thus provides a mechanism where actuation of the elastic element 120 shortens or widens the distance between the ends of the expandable basket 118, thereby expanding or collapsing the structure.

In other embodiments, actuating means (not shown), such as magnetic, thermal, electrical, or mechanical, may replace the elastic element 120. Magnetic means may include electromagnetic means, wherein repulsive or attractive forces actuate the movable attachment point. Electrical heating of fluids may also provide actuation due to expansion of fluids. Thermal energy may also act on shape memory alloys to provide actuation.

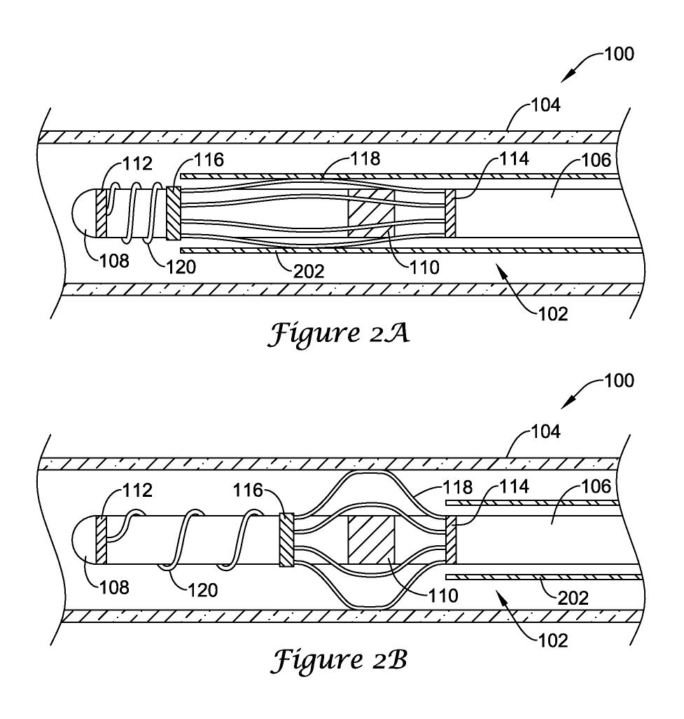

The expandable basket 118 may further be collapsed or expanded by physical means, such as by the use of a sheath 202 (see FIGS. 2A and 2B), or by manual actuation by use of pull wires such as pull wire 122 (see FIGS. 1A and 1B) or pull wire 302 (see FIGS. 3A and 3B). FIGS. 2A and 2B illustrate a cross section of the expandable basket 118 in collapsed and expanded positions, through introduction and withdrawal of the sheath 202, respectively. The sheath 202 may take the form of an elongated tubular member, which may be slipped over the catheter shaft 106.

In some embodiments, the elastic element 120 may be biased to take a compressed configuration, such as that shown in FIG. 1A, and pull wire 122 may be pulled proximally against the force of the elastic element 120 to open basket 118 to the expanded position shown in FIG. 1B. When pull wire 122 is released, the elastic element 120 may return to the position shown in FIG. 1A to collapse the basket 118. Alternatively, the elastic element 120 may be biased to take an expanded or elongated configuration such as that shown in FIG. 1B, and pull wire 122 may be pushed distally against the force of the elastic element 120 to collapse the basket 118. When pull wire 122 is released, the elastic element 120 may return to the position shown in FIG. 1B to expand the basket 118.

Another actuation method is illustrated with respect to FIGS. 2A and 2B. In embodiment of FIGS. 2A and 2B, a sheath 202 is positioned over the expandable basket 118 at the distal end 108 portion of the catheter shaft 106. The sheath 202 and the catheter may be configured to advance through a vasculature as a unit. Moreover, the sheath 202 may be translatable along the catheter shaft 106 from a first position, in which the expandable basket 118 is within the sheath 202, to a second position, in which the expandable basket 118 is no longer within the sheath 202. The exposed expandable basket 118 then expands to the position shown in FIG. 2B due to force exerted by the elastic element 120. The sheath 202 may then be advanced over the basket to force the basket back into the collapsed position shown in FIG. 2A. In some embodiments, the sheath 202 may frictionally engage the catheter shaft 106 when the sheath 202 is translated along the catheter shaft 106.

FIGS. 3A and 3B illustrate an alternate embodiment where the elastic element 120 is actuated between its expanded state and contracted states by use of pull wire 302. In this embodiment, expandable basket 118 is distal the elastic element 120, and the expandable basket 118 is attached to the fixed first attachment point 112 towards the distal end and the collar 116 towards the proximal end. The collar 116 is attached to the pull wire 302. The pull wire 302 may extend through a separate lumen (not shown) in the catheter shaft 106, and contact the movable attachment point 116 towards its center. The separate lumen has a longitudinal slit at its distal end to allow for the proximal and distal movement of the collar 116 over the catheter. Alternatively, the collar 116 may be affixed to the pull wire 302 as shown in the figure, and the pull wire may traverse the vasculature outside the catheter shaft 106.

In some embodiment, the elastic element 120 may be biased to its expanded state and the pull wire 302 may be urged proximally to shift/hold the elastic element in compression to keep the basket 118 in a collapsed state. When deployment of the basket 118 is desired, the pull wire 302 may be released to allow the elastic element 120 to expand to push collar 116 distal, which in turn expands the basket 118. Alternatively, the elastic element 120 may be biased to its compressed state and the pull wire 302 may be urged distally to expand the basket 118.

Further, the proximal end of the elongate shaft may include a hub attached thereto for connecting other treatment devices, or for providing a port for facilitating other treatments. The catheter may further include one or more lumens extending there through. For example, the catheter may include a guidewire lumen and/or one or more inflation lumens. The lumens may be configured in any way known in the art. For example, the guidewire lumen may extend the entire length of the elongated shaft, such as in an over-the-wire catheter, or may extend only along a distal portion of the elongated shaft, such as in a single operator exchange (SOE) catheter.

These examples are not intended to be limiting, but rather examples of some possible configurations. While not explicitly shown, the system 100 may further include temperature sensors, wire, an infusion lumen, radiopaque marker bands, fixed guidewire tip, a guidewire lumen, external sheath and/or other components to facilitate the use and advancement of the system 100 within the vasculature may be incorporated.

It should be apparent that the medical device of the present disclosure may be used to carry out a variety of medical or non-medical procedures, including surgical and diagnostic procedures in a wide variety of bodily locations. For example, mucosal resection, or ablation, polyp removal, or of a variety of body organs, such as esophagus, stomach, bladder, or the urethra could be accomplished using the method discussed above. In addition, at least certain aspects of the aforementioned embodiments may be combined with other aspects of the embodiments, or removed without departing from the scope of the disclosure.

Other embodiments of the present disclosure will be apparent to those skilled in the art from consideration of the specification and practice of the embodiments disclosed herein. It is intended that the specification and examples be considered as exemplary only, with a true scope and spirit of this disclosure being indicated by the following claims.

What is claimed is:

1. A catheter system for insertion in a patient's vasculature, the system comprising:
a catheter having a proximal end, and a distal end;
an electrode for tissue modulation disposed on a distal portion of the catheter;
an expandable basket structure placed over a distal section of the catheter, the expandable basket structure having:
a basket having a fixed end and a movable end, wherein the movable end is slidably coupled to the catheter; and
an actuating structure connected to the movable end for shifting the basket between a collapsed state and an expanded state.

2. The system of claim 1, wherein the basket is proximal the actuating structure.

3. The system of claim 1, wherein the basket is distal the actuating structure.

4. The system of claim 1, wherein the actuating structure is a spring.

5. The system of claim 1, further comprising a pull wire attached to and extending proximally from the movable end of the basket.

6. The system of claim 1, further comprising a sheath extending over the basket to retain the basket in the collapsed state, the sheath movable proximally relative to the catheter.

7. The system of claim 1, wherein the basket expands symmetrically.

8. The system of claim 1, wherein the basket expands asymmetrically.

9. The system of claim 1, wherein the basket is disposed over the electrode.

10. The system of claim 1, wherein the electrode is cylindrical and surrounds a portion of catheter.

11. The system of claim 1, wherein the electrode is an ablation electrode.

12. The system of claim 1, wherein the basket comprises a non-metallic material.

13. A catheter system for insertion in a patient's vasculature, the system comprising:
a catheter shaft having a distal end region;
an ablation electrode disposed along the distal end region of the shaft;
an expandable basket disposed over the ablation electrode, the expandable basket including a plurality of struts that each include a first end attached to a fixed attachment member and that each include a second end attached to a collar;
wherein the collar is slidable along the catheter shaft;
a spring attached to the collar; and
a pull wire attached to the collar, the pull wire being configured to shift the basket between a collapsed configuration and an expanded configuration.

14. The system of claim 13, wherein the spring is biased to be in a compressed configuration and wherein actuating the pull wire overcomes the bias of the spring and shifts the spring to an elongated configuration.

15. The system of claim 14, wherein actuating the pull wire shifts the basket to the expanded configuration.

16. The system of claim 13, wherein the spring is biased to be in an elongated configuration and wherein actuating the pull wire overcomes the bias of the spring and shifts the spring to a compressed configuration.

17. The system of claim 16, wherein actuating the pull wire shifts the basket to the collapsed configuration.

18. A method for ablating nerve tissue adjacent a renal artery, the method comprising:
advancing an ablative catheter intravascularly to a location proximal of a nerve, the ablative catheter including a catheter shaft, an electrode and an expandable non-metallic basket extending over a section of the catheter;
deploying the basket into an expanded position in a vessel lumen such that portions of the basket contact a wall of the vessel lumen, and the electrode maintains a distance from the wall of the vessel lumen; and
activating the electrode to ablate at least a portion of the nerve.

19. The method of claim 18, wherein deploying the basket includes releasing a pull wire held in tension.

20. The method of claim 18, wherein deploying the basket includes moving a sheath proximally from over the basket.
